(19) United States
(12) Patent Application Publication
(10) Pub. No.: US 2005/0038326 A1
(43) Pub. Date: Feb. 17, 2005

(54) System, device, and method for remote monitoring and servicing

(76) Inventor: Michael Mathur, Franklin, MA (US)

Correspondence Address: Jeffrey T. Klayman;Bromberg & Sunstein LLP, 125 Summer Street, Boston, MA, 02110-1618, US

(21) Appl. No.: 10/856,744

(22) Filed: May 28, 2004

Related U.S. Application Data
(60) Provisional application No. 60/474,790, filed on May 30, 2003.
(60) Provisional application No. 60/478,491, filed on Jun. 13, 2003.

(51) Int. Cl. A61B 5/00

(52) U.S. Cl. 600/300; 340/573.1

(57) ABSTRACT

A system, device, and method for remote monitoring and servicing uses a gateway to collect user information, such as physiological, audio, video, and proximity information. The gateway processes the user information locally, and may send the user information or other information, such as alarms, to remote service providers through one or more head-end servers. The gateway also allows service provider information, such as audio and video information, to be conveyed from the service providers to the users. The gateway may output video information on a predetermined television channel, and may output audio information on the predetermined television channel or to a wireless remote controller with in-built speaker. The gateway may receive audio information from the user via a wireless remote controller with in-built microphone. The gateway provide for videoconferencing between the user and one or more remote service providers.

### PRIORITY

The present invention claims priority from the following United States provisional patent applications, which are hereby incorporated herein by reference in their entireties:
Application No. 60/474,790, filed on May 30, 2003; and
Application No. 60/478,491, filed on Jun. 13, 2003.

### FIELD OF THE INVENTION

The present invention relates generally to communication systems, and more particularly to a system, device, and method for remote monitoring and servicing.

### BACKGROUND OF THE INVENTION

Studies have shown that the population of the United States and other countries is aging, with the number of people aged 65 and above expected to increase both in absolute terms and as a percentage of overall population. The number and severity of health problems, and particularly chronic conditions, generally increase with age, with an estimated 69 percent of those over the age of 65 having more than one chronic condition and over 50 percent of those between the ages of 45 and 64 having more than one chronic condition. As a result, the economic burden of chronic diseases in the United States is expected to increase from approximately 100 billion dollars a year now to approximately 900 billion dollars a year in 2050. Overall, national health expenditures is expected to grow both as a percentage of gross domestic product and per capita. A large proportion of the health expenditures is due to emergency room visits and hospitalizations.

At the same time, the number of health care workers is declining relative to the population. This may be the result of a number of factors, including the high cost of medical school, the many years of schooling and training to become a doctor, the burdensome cost of practicing medicine (e.g., malpractice insurance), and the drive by health maintenance organizations to lower the amounts paid to doctors and hospitals. The number of geriatricians in particular is decreasing, while the number of geriatric patients is increasing, making it particularly difficult for the elderly to receive adequate health care.

### SUMMARY OF THE INVENTION

In an embodiment of the present invention there is provided a method of monitoring an individual in a first location that involves sensing at least one physiological parameter of the individual and providing at least one parameter output, providing information related to the at least one parameter output over a communication link to at least one monitoring facility in at least one location different from the first location (such as, for example, providing a first parameter output to a first monitoring facility and providing a second parameter output to a second monitoring facility), and using the communication link to permit video information derived from the first location to be conveyed to a monitoring facility and information including at least audio information derived from the monitoring facility to be conveyed to the first location. The communication link may also be used to permit video information derived from the monitoring facility to be conveyed to the first location. The method may also involve determining automatically at a monitoring facility that at least one parameter output reaches an alarm condition (such as, for example, determining that the at least one parameter output indicates a likelihood for a predetermined disease, e.g., SARS, smallpox, influenza, multi-drug resistant tuberculosis, congestive heart failure, asthma, diabetes, chronic obstructive pulmonary disease, heart attack, stroke, and seizure, to name but a few) and providing a parameter alarm indicating the alarm condition. The parameter alarm may be provided, for example, by placing a phone call, lacing a page to a paging device, communicating with a mobile phone, or communicating the proximity alarm over a digital communication network.

In another embodiment of the present invention there is provided a method of monitoring an individual in a first location that involves sensing at least one physiological parameter of the individual and providing at least one parameter output, providing information related to the at least one parameter output over a communication link to at least one monitoring facility in at least one location different from the first location (such as, for example, providing a first parameter output to a first monitoring facility and providing a second parameter output to a second monitoring facility), and using the communication link to permit information including at least audio information derived from the first location to be conveyed to a monitoring facility and video information derived from the monitoring facility to be conveyed to the first location. The communication link may also be used to permit video information derived from the first location to be conveyed to the monitoring facility. The method may also involve determining automatically at a monitoring facility that at least one parameter output reaches an alarm condition (such as, for example, determining that the at least one parameter output indicates a likelihood for a predetermined disease, e.g., SARS, smallpox, influenza, multi-drug resistant tuberculosis, congestive heart failure, asthma, diabetes, chronic obstructive pulmonary disease, heart attack, stroke, and seizure, to name but a few) and providing a parameter alarm indicating the alarm condition. The parameter alarm may be provided, for example, by placing a phone call, lacing a page to a paging device, communicating with a mobile phone, or communicating the proximity alarm over a digital communication network.

In another embodiment of the present invention there is provided a method of providing remote conference capability to an individual at a first location that involves using a television device at the first location, coupled through an interface to at least one communication link, to convey audio and/or video information from a second location to the individual and using a remote controller equipped with a microphone at the first location to direct operation of the interface and to provide an audio input to the interface. The audio information derived from the audio input is transmitted to the second location over the at least one communication link.

In order to use the television device at the first location to convey audio and/or video information to the individual, the audio and/or video information is typically received by the interface from a second location over the at least one communication link and transmitted by the interface to the television device, for example, on a predetermined television channel. The audio and/or video information may include such things as medical information (such as a reminder to take medication, first aid information, home remedy information, medicine information such as side-effects and precautions, surgery recovery information, or infection disease management information), educational information (such as lecture materials, remote testing, parent-teacher videoconferencing, or automated student reporting), advertising information (including local, regional, state, national, or global advertising information), weather information (including local, regional, state, national, or global weather information), or energy management information (such as a power meter reading, a water meter reading, a gas meter reading, energy efficiency information, energy cost information, or energy manufacturer selection options), to name but a few. At least some of the audio and/or video information conveyed to the individual may be selectable by the individual using the remote controller. At least some of the audio and/or video information may be conveyed to the individual based on a profile stored in the interface. The user's profile may include private information (such as, for example, diseases, medical conditions, medications, allergies), and this local storage and management of the user's profile helps to keep such information private while allowing it to be used to filter information presented to the user.

The method may further involve using a video camera to capture video information at the first location and transmitting the video information by the interface to a second location over the at least one communication link. The video information may be communicated to the interface in any of a variety of ways. For example, the video camera may be coupled directly to the interface or the video camera may be coupled or integral to the remote controller (in which case the video information is transmitted by the remote controller to the interface).

A remote conference may be initiated from either the first location or the second location.

In one exemplary embodiment, the conference is initiated from the second location, for example, by the interface receiving an incoming signal from the second location over the at least one communication link indicating the start of a conference and activating the conference in response to the incoming signal. As part of activating the conference, the interface may transmit an alert signal to the remote controller in response to the incoming signal. The remote controller may, in turn, generate an alert to the individual in response to the alert signal in any of a variety of ways. For example, the remote controller may include an audio output device (such as a speaker or buzzer), in which case the remote controller may produce an audible sound from the audio output device to alert the individual. The remote controller may alternatively or additionally include a light emitting device (such as an LED), in which case the remote controller may activate the light emitting device to alert the individual. The remote controller may alternatively or additionally include a text display (such as an LCD display), in which case the remote controller may display a text message on the visual display to alert the individual.

In another exemplary embodiment, the conference is initiated from the first location using the remote controller. In this case, the remote controller typically transmits an alert signal to the interface indicating the start of a conference (for example, when a predetermined button on the remote controller is depressed by the individual), and the interface activates the conference in response to the alert signal. The interface may activate the conference in any of a variety of ways, such as, for example, transmitting an outgoing signal to the second location over the communication link in response to the alert signal.

As part of any conference, the interface may cause the television to be turned on (if not already on) and tuned to a predetermined television channel.

An embodiment of the present invention may also support an “always on” form of conferencing in which audio and/or video information is streamed from the first location to the second location and/or from the second location to the first location and conveyed to the user over a predetermined television channel. If a user tunes to the appropriate channel, then the user receives the audio and/or video from the remote location. This can be done without notifying anyone at the remote location or with notifying the remote location, for example, by transmitting a signal over the at least one communication link. This would allow someone at one location, for example, so selectively monitor an individual at the remote location by simply tuning in to the appropriate channel and changing the channel (or turning off the television) to stop monitoring. In order to support this type of functionality, the interface may include a “block/add” capability in which a programmable filter blocks out the predetermined television channel (e.g., from cable, satellite, or antenna) and a programmable transmitter inserts a television signal on that television channel including the audio and/or video information from the remote location.

In embodiments of the present invention, the at least one communication link may include a persistent communication link allowing communication with the second location. The interface may include redundant communication interfaces to a plurality of communication links. The interface may alternatively or additionally include a first communication interface to a first communication link and a second communication interface to a second interface coupled to a second communication link. The at least one communication link may include communication over the Internet or a private intranet. The at least one communication link may include any of a variety of communication connections, such as cable modem, digital subscriber line (DSL), dial-up mode, or wireless modem.

The conferencing method may additionally involve sensing at least one of a physiological parameter of the individual, a proximity of the individual, and a location-specific parameter, providing a parameter output to the interface, and providing information related to the parameter output by the interface over a communication link to at least one monitoring facility in at least one location different from the first location.

The conferencing method may additionally involve sensing a location-specific parameter, providing a parameter output to the interface, and conveying audio and/or video information to the individual using the television device based on the parameter output.

In another embodiment of the present invention there is provided a remote controller for a conferencing system. The remote controller includes a microphone for receiving audio information and a wireless communication interface for at least transmitting the audio information to a conferencing device.

In another embodiment of the invention there is provided a remote monitoring system including an interface situated at a first location; a server situated at a second location; a communication link between the interface at the first location and the server at the second location; a television device, situated at the first location and in communication with the interface, for conveying audio and/or video information, received by the interface from the server over the communication link, to an individual at the first location; and a remote controller, the remote controller being equipped with a microphone and being in wireless communication with the interface, for directing operation of the interface and providing an audio input to the interface, the interface transmitting audio information derived from the audio input to the server over the communication link.

In another embodiment of the invention there is provided a method of monitoring an individual who is subject to detention in a first location. The method involves sensing at least one physiological parameter of the individual and providing a parameter output, sensing the proximity of the individual and providing a proximity output, and providing information related to the parameter output and the proximity output over a communication link to at least one monitoring facility in at least one location different from the first location.

In a related embodiment, the method may also involve using the communication link to permit video information derived from the first location to be conveyed to a monitoring facility and information including at least audio information derived from the monitoring facility to be conveyed to the first location. In a further related embodiment, using the communication link may further involve using the communication link to permit video information derived from the monitoring facility to be conveyed to the first location.

Another related embodiment of the invention may further involve determining automatically at a monitoring facility that the proximity output reaches an alarm condition, and providing a proximity alarm indicating the alarm condition. In a further related embodiment, determining automatically at the monitoring facility may involve determining that the individual is outside the range of a proximity sensor. In the alternative, determining automatically at the monitoring facility may involve determining that the individual has removed a proximity device. In another further related embodiment, providing a proximity alarm may involve at least one of automatically placing a phone call, placing a page to a paging device, communicating with a mobile phone, and communicating the proximity alarm over a digital communication network.

In another related embodiment, the method may involve determining automatically at a monitoring facility that the parameter output reaches an alarm condition, and automatically providing a parameter alarm indicating the alarm condition. Determining that the parameter output reaches an alarm condition may involve determining that the parameter output indicates a likelihood for a predetermined disease, such as SARS, smallpox, influenza, multi-drug resistant tuberculosis, congestive heart failure, asthma, diabetes, chronic obstructive pulmonary disease, heart attack, stroke, and seizure, to name but a few. Providing a parameter alarm may involve at least one of placing a phone call, placing a page to a paging device, communicating with a mobile phone, and communicating the proximity alarm over a digital communication network.

In still another related embodiment of the invention, providing information may involve providing the information related to the parameter output to at least one parameter monitoring facility, and providing the information related to the proximity output to at least one proximity monitoring facility.

### BRIEF DESCRIPTION OF THE DRAWINGS

In the accompanying drawings:

FIG. 1 is a block diagram showing various components of an exemplary remote services and monitoring system in accordance with an embodiment of the present invention;

FIG. 2 is a block diagram showing various components of an exemplary remote location in accordance with an embodiment of the present invention;

FIG. 3 shows additional details of the components at a remote location in accordance with an embodiment of the present invention;

FIG. 4 is a block diagram showing two interfaces and interconnected in a redundant configuration over a high-speed redundant communications link in accordance with an embodiment of the present invention;

FIG. 5 shows an exemplary remote controller in accordance with an embodiment of the present invention;

FIG. 6 shows an exemplary audio and/or video conferencing system in which the television is used for conveying audio and/or video information to an individual and the remote controller with built-in microphone is used for conveying audio information from the individual, in accordance with an embodiment of the present invention;

FIG. 7 shows an exemplary set of hardware/software components designed to be used in the quarantine location in accordance with an embodiment of the present invention;

FIG. 8 shows an exemplary set of hardware/software components designed to be used in a monitoring facility remote from the quarantine location in accordance with an embodiment of the present invention; and

FIG. 9 shows an exemplary protocol stack for a television interface gateway in accordance with an embodiment of the present invention.

### DETAILED DESCRIPTION OF A PREFERRED EMBODIMENT

Embodiments of the present invention provide for remote servicing and monitoring over a communication network. Specifically, various service provider locations are in communication with various remote locations over the communication network. Audio, video, and other types of information can be exchanged among and between the various service provider and remote locations. A television may be used to convey audio and/or video information to an individual. A wireless remote controller with a built-in or external microphone may be used as an audio input device for conferencing, recording, and/or voice activated control and communications. The wireless remote controller may include a bi-directional communication interface for both sending and receiving signals. The wireless remote controller may include various output devices, such as LEDS, a buzzer, a speaker, and/or a display, that can be used for such things as alerting the individual and providing audio, video, textual, and graphical information to the individual.

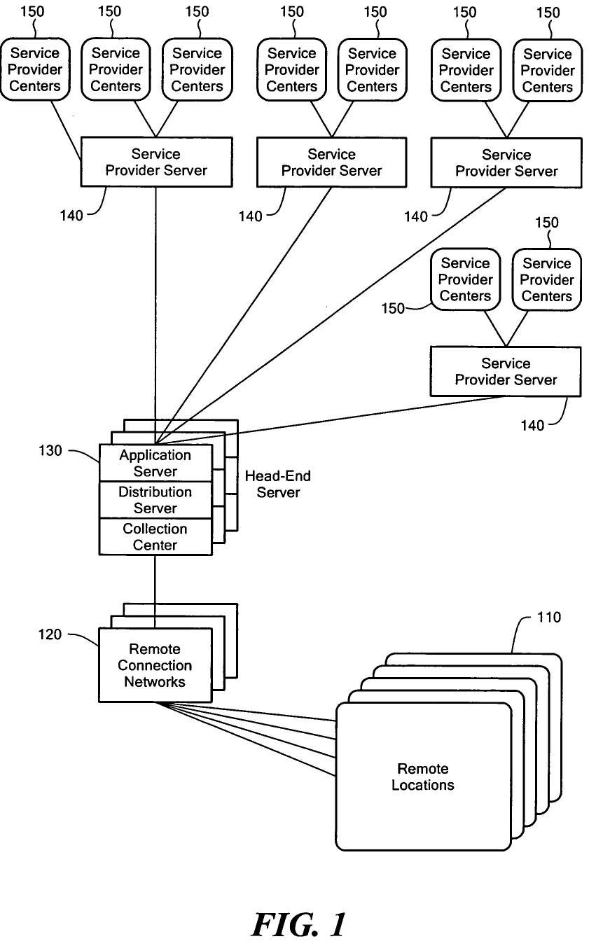

FIG. 1 is a block diagram showing various components of an exemplary remote services and monitoring system in accordance with an embodiment of the present invention. Among other things, the system includes remote locations 110 to be remotely serviced and monitored. The remote locations 110 can include such things as homes, apartments, and hotel rooms, to name but a few. The remote locations 110 are connected over various types of communication links (e.g., broadband or POTS links, Ethernet networks, or wireless networks, to name but a few) to remote connection networks 120. At least one of the communication links is typically an “always on” communication link. The remote connection networks 120 can include the Internet, a private intranet, or other types of communication networks.

Information collected at the remote locations 110 is sent to a head-end server 130 through the remote connection networks 120, for example, in response to a request for information made by a service provider through a service provider server 140. The head-end server 130 typically includes a collection server, a distribution server, and an application server which process the information, prepare it for distribution to the service provider servers 140, and analyze it to generate reports and alarms based on pre-configured settings. The head-end server 130 interactively communicates with the remote locations 110 via the remote connection networks 120.

The head-end server 130 distributes the information to the appropriate service provider server(s) 140, which in turn communicate with various service provider centers 150 over networks including local area network or Internet. The service provider centers 150 communicate with the service provider servers 140 and the head-end server 130 interactively using logins with authentication and other secure communications.

Interactive communication between the service provider centers 150 and the remote locations 110 is enabled through the remote communication networks 120, the head-end server 130, and the service provider servers 140. The head-end server 130 and/or the remote connection networks 120 also typically allow interactive communication between different remote locations 110 and/or between different service providers over local or wide area networks or Internet.

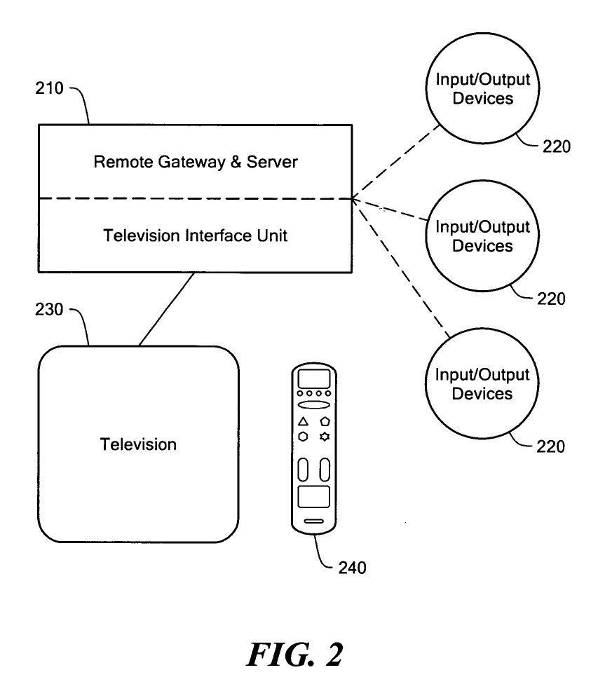

FIG. 2 is a block diagram showing various components of an exemplary remote location in accordance with an embodiment of the present invention. Among other things, remote location 110 includes an interface 210 and various devices in wired or wireless communication with the interface 210, such as a television 230 (e.g., for conveying video and/or audio information to an individual), a wireless remote controller 240 (e.g., for inputting information to the interface 210 and possibly also for receiving information from the interface 210), and various other input/output devices 220 (e.g., a video camera for providing a video input to the interface 210, a microphone for providing an audio input to the interface 210, medical equipment for monitoring and/or providing medical care to an individual, etc.).

The interface 210 is in communication with the remote connection networks 120 through one or more communication links, and is also in communication with the various input/output devices 220, the television 230, and the wireless remote controller 240 through wired and/or wireless communication links. Communication with the remote connection networks 120 is typically bi-directional, while communication with the various devices within the remote location 110 may be unidirectional or bi-directional depending on the various device capabilities.

The input/output devices 220 can include any of a variety of device types. For visually monitoring an individual at the remote location 110, for example, the input/output devices 220 may include a video camera that transmits video information to the interface 210. For medically monitoring an individual, for example, the input/output devices 220 may include various types of medical devices that obtain physiological information from the individual and transmit the physiological information to the interface 210, such as a blood pressure monitor, a temperature monitor, or a blood sugar monitor, to name but a few. The system may provide for remote operation, calibration, and maintenance of certain input/output devices by the service providers.

The television is typically used for, among other things, conveying video and/or audio information to an individual at the remote location 110. The interface 210, and in particular, the television interface unit of the interface 210, can block a predetermined channel and insert video and/or audio information on a predetermined television channel to which the individual can tune to receive the video and/or audio information. The video and/or audio information might include information sent from a service provider center 150, such as, for example, medical information or instructions, might include information obtained from within the remote location 110, or might include videoconferencing information from one or more remote locations. The television 230 can also be used to receive the video and/or audio component of a video and/or audio conference.

The interface 210 is capable of collecting various types of information from the input/output devices 220 and the remote controller 240, which it can transmit to the head-end server 130, display on the television 120, and/or process locally. The interface 210 is also capable of receiving various types of information from the head-end server 130, which it can process locally and/or transmit to the television 230, the input/output devices 220, and/or the remote controller 240. The interface 210 typically stores various pre-configured settings (profiles) that it uses to decide, among other things, what information from the remote location 110 to transmit to the head-end server 130 and/or what information from the head-end server 130 to present to an individual at the remote location 110 (e.g., via the television 230, the input/output devices 220, and/or the remote controller 240).

In a typical monitoring scenario, the interface 210 collects information from the various input/output devices 220 (such as medical monitoring equipment) and transmits the information to the head-end server 130. This information may be streamed, transmitted periodically, transmitted upon a request from the head-end server 130, or transmitted under other circumstances (such as, for example, when an alarm condition occurs).

In a preferred embodiment of the present invention, the remote controller 240 includes a bi-directional wireless communication interface for bi-directional communication with the interface 210. The remote controller 240 preferably also includes an integral microphone and/or an external microphone connection for providing an audio input to the interface 210. Among other things, the audio input can be used to allow for remote conferencing with one or more person(s) at a service provider center 150 and/or other remote locations and/or for voice-activated control of the interface 210. The remote controller 240 preferably also includes one or more output devices, such as a buzzer or speaker, various LEDs, and/or a text display screen. The interface 210 can send instructions to the remote controller 240 to activate and control these output devices, for example, to alert an individual of some important event.

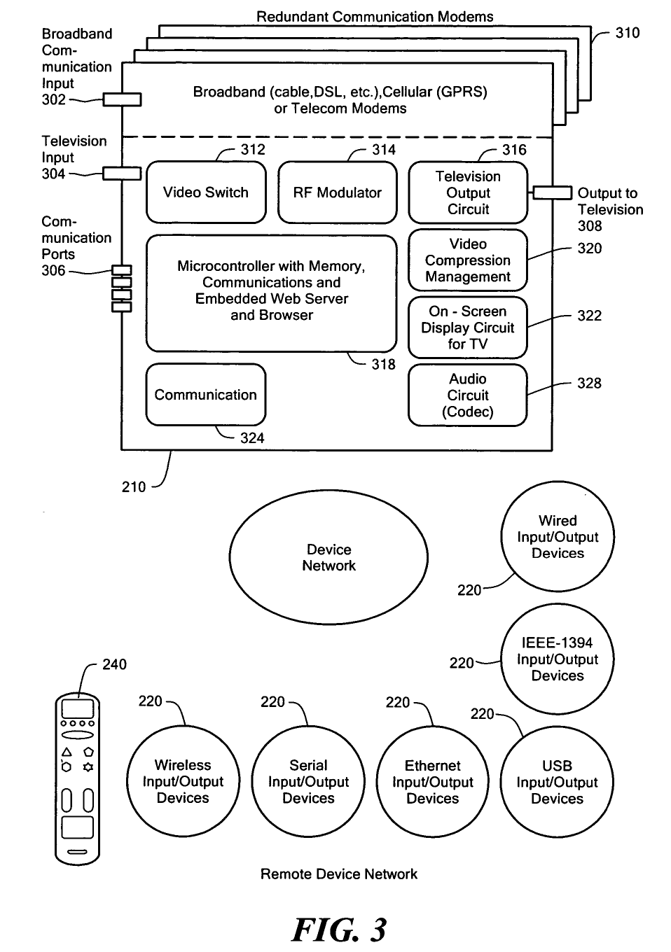

FIG. 3 shows additional details of the components at a remote location 110 in accordance with an embodiment of the present invention. As shown in FIG. 3, the interface 210 typically includes a broadband/communication input 302 for connection to one or more communication networks, a television input 304 for receiving television input signals (e.g., from an optional set-top box, antenna, cable, or satellite dish), a television output 308 for outputting television signals (e.g., to an optional set-top box, to the television 230, or to some other device such as a VCR or DVD player/recorder), and various communication ports 306 for communicating with local input/output devices 220.

The interface 210 is typically a microprocessor based system having a microcontroller with memory 318 that supports, among other things, communications, a web server, and a web browser). The interface 210 typically also includes a video switch 312, an RF modulator 314, television output circuitry 316, video compression circuitry 320, on-screen display circuitry 322, an audio codec 328, and communication circuitry 324 including a configurable channel blocking circuit. The interface 210 preferably communicates bi-directionally with the wireless remote controller 240 through the wireless communication circuitry 324.

The interface 210 can support a variety of communication technologies (e.g., wireless such as 802.11b/g and/or infrared, serial such as RS232C, Ethernet, USB, Firewire, power line, etc.) and a variety of control mechanisms (e.g., master/slave, client/server, unicast/multicast/broadcast, token passing, synchronous/asynchronous, etc.). The interface 210 can support streaming audio and video. The interface 210 can support such things as automatic recognition, address, and configuration of devices.

The interface 210 typically includes an operating system, network functions, windowing software, a web browser, and a web server. The interface 210 can output various types of video and/or audio signals through the television output, including, but in no way limited to, regular television signals from the television input, the output of the web browser, the output of the windowing software, video and/or audio information received over the broadband/communication input, video and/or audio information received from local input/output devices 220, and video and/or audio signals generated internally by the interface 210. The interface 210 can select the television output based on a number of factors, including, but in no way limited to, information received from the head-end server 130, information received from the local input/output devices 220, and information received from the wireless remote controller 240. The interface 210 can preferably insert video and/or audio information into an output television channel (e.g., display text or graphics messages on the television 230 when a regular television channel is being displayed). The interface 210 generally passes all channels from the television input 304 to the television output 308 when it is turned off (e.g. powered down) and all channels except the predetermined and blocked channel when is turned on (e.g. powered on). The interface 210 can be powered on or off using either the remote controller 240 or a mechanical switch on the interface 210.

The inbuilt web-browser also allows the interface 210 to display information collected from local devices 220 over the communications ports or received from the head-end server 130. The remote controller 240 preferably operates as a general purpose input/output device as well as a navigation device for the television 230 display.

As shown in FIG. 3, the interface 210 preferably includes one or more integral communication modems 310, such as a cable modem, a DSL modem, a wireless modem, or a telecom modem. The modems 310 are preferably modular and can be added or removed as desired. Multiple modems (using same and/or different technologies) can be used to provide communication redundancy and fault tolerance. The interface 210 may additionally or alternatively include communication ports for connecting to external modems and/or other interfaces 210. The interface 210 preferably tests the communication links periodically and dynamically decides and selects primary and backup paths for communications with the head-end server 130. The primary and backup communications paths can be also selected by a pre-configured option or by service providers 150 via the head-end server 130 and the remote communication network 120.

As described above, the interface 210 is preferably in bi-directional communication with the remote controller 240, and the remote controller 240 preferably includes a microphone and/or various output devices. The interface 210 can set signals to the remote controller 240 to control the output devices (e.g., turn a LED on/off, sound a buzzer, send an audio stream to a speaker, etc.). The interface 210 can also receive signals from the remote controller 240, preferably including audio signals (e.g., from an integral or attached microphone).

As shown in FIG. 3, the interface 210 typically includes video compression firmware and circuitry 320. Among other things, this circuitry allows the interface 210 to encode and decode video signals in real time.

In various embodiments of the present invention, multiple interface devices are interconnected to allow for redundancy. In such a configuration, an interface can communicate with the head-end server 130 through one or more other interfaces, should such communication paths become necessary or desirable, for example, due to failure or congestion of direct communication links to the head-end server 130.

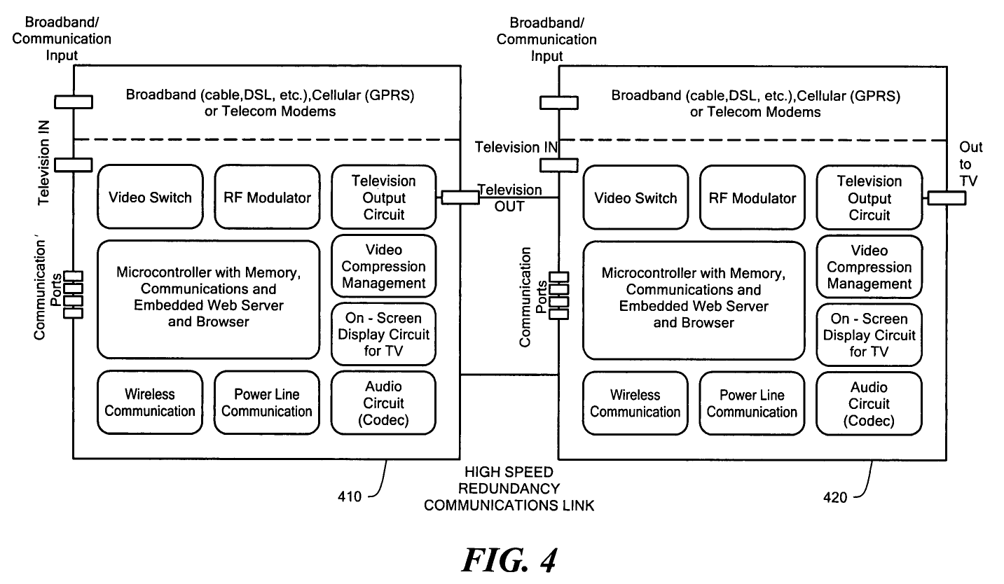

FIG. 4 is a block diagram showing two interfaces 410 and 420 interconnected in a redundant configuration over a high-speed redundant communications link in accordance with an embodiment of the present invention. This redundancy allows both interfaces 410 and 420 to communicate with the head-end server 130 directly over same or different broadband communications links, should such communication paths become necessary or desirable, for example, due to failure or congestion of direct communication links between the interfaces 410 or 420 and the head-end server 130.

Each interface 410 and 420 typically tests all communication paths to the head-end server 130 (including direct communication links and communication links through other interfaces) and selects the best communication path(s) for communicating with the head-end server 130. The interfaces 410 and 420 typically transmit the test information to the head-end server 130, and the head-end server 130 can use the test information to select primary and backup communication paths to the interfaces 410 and 420. Communication link selections can also be made by an individual through the remote controller 240 or though a pre-configured selection or online by a service provider 150.

FIG. 4 shows a feature of the redundant configuration in which television connections are essentially “daisy-chained” across multiple redundant interfaces. Specifically, the television output of interface 410 can be connected to the television input of interface 420, which allows interface 420 to, among other things, either pass the television signal from interface 410 through to the television output of interface 420 or block the television signal from interface 410 and pass a different television signal through to the television output of interface 420. This blocking and insertion of television signals can be done on a channel-by-channel basis, such that, for example, a single television channel can be blocked at the television input and different television signal can be inserted on the same television channel at the television output.

In a typical redundant configuration, one of the interfaces will be the “primary” interface for controlling the remote location during normal operation, and the other interface(s) will be “backup” interfaces for controlling the remote location if the primary interface fails. For example, in FIG. 4, the interface 410 may be the primary interface and the interface 420 may be a backup interface. During normal operation, the interface 410 communicates with the interface 420 over the high-speed redundancy communications link to provide current information to the interface 420. If the interface 410 fails, the interface 420 takes over controlling the remote location. This switch-over is done automatically by the interface 420 based on the status of the interface 410.

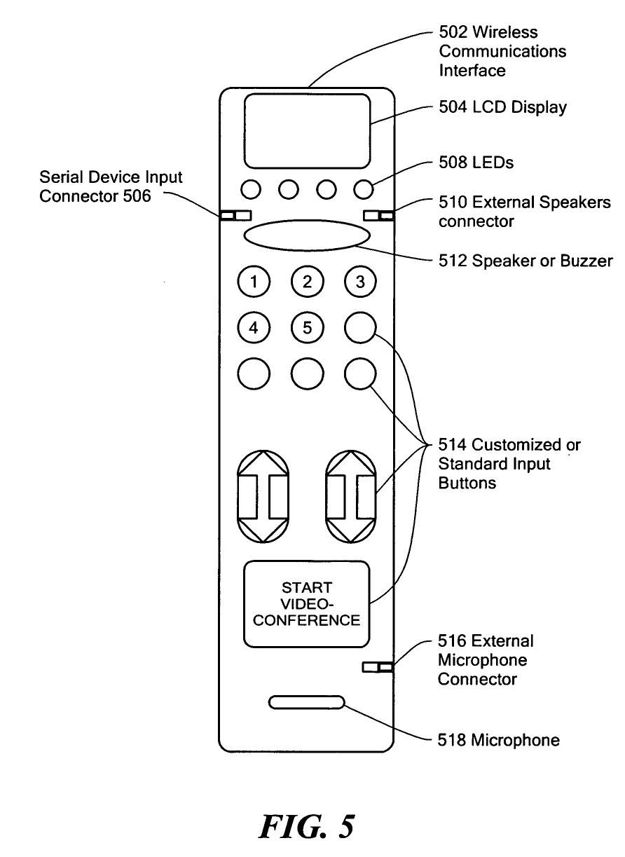

FIG. 5 shows an exemplary remote controller 500 in accordance with an embodiment of the present invention. Among other things, the remote controller 500 can include such things as a wireless communication interface 502 (e.g., IR or RF), a LCD display 504, a serial device input connector 506, LEDs 508, an external speaker connector 510, a speaker or buzzer 512, customizable and/or standard input buttons 514, an external microphone connector 516, and a built-in microphone 518. Certain buttons 514 may be customized for particular applications, and other buttons 514 may operate “standard” control functions (e.g., number keys, up/down arrows, TV, VCR, DVD controls, etc.). The remote controller 500 is typically battery operated, and may include a rechargeable battery.

As discussed above, the wireless communications interface 502 is preferably a bi-directional interface that allows for bi-directional communication with the interface 210 and possibly also with other devices. The LCD display 504 can be used to display text and/or graphics.

The serial device connector 506 can be used to connect a serial device to the remote controller 500 (e.g., through an RS232 interface). The serial device can be controlled directly by the remote controller 500 or remotely by the interface 210 through the remote controller 210. The remote controller 500 can also enable communication between the interface 210 and the serial device.

The LEDs 508 can be used for local indications and alerts. The LEDs can be remotely controlled by other devices, such as the interface 210, for example, through the wireless communication interface 502.

The speaker or buzzer 512 can be used to generate an audible alert, for example, upon receiving an alert signal from another device, such as the interface 210 over the wireless communication interface 502. Depending on the type of speaker or buzzer, different types alerts can be used for different types of events.

Alternatively, or additionally, the speaker or buzzer 512 can be used to play audio information, for example, based on audio signals received from another device, such as the interface 210 over the wireless communication interface 502. It should be noted that the remote controller 500 can alternatively or additionally send alert and/or audio signals to an external speaker over the external speaker connection 510.

The remote controller 500 can transmit audio signals from the microphone 518 or external microphone connector 516 to another device, for example, over the wireless communication interface 502. The remote controller 500 can send the audio signals as a modulated signal or as a digitized signal.

Among other things, the ability of the remote controller 500 to transmit audio information allows it to be used for audio and/or video conferencing. For example, in an exemplary embodiment of the present invention, the remote controller 500 with integral microphone can be used as the audio input for a conference, and the television can be used as the audio and video output for a conference. Audio signals from the built-in microphone are sent by the remote controller 500 to the interface 210, which in turn forwards audio information to a remote conferencing site over the communication links. Audio and/or video information received by the interface 210 over the communication links are output through the television output on a predetermined television channel to be played on the television. A video camera can be used at the remote location to provide video signals to the interface 210, which in turn transmits video information to the remote conferencing site over the communication links.

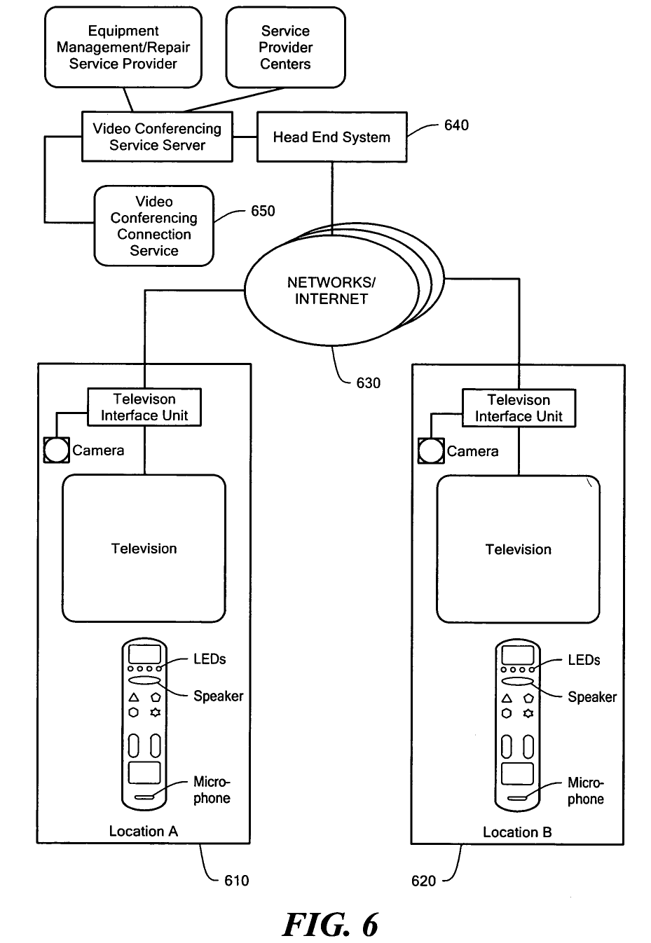

FIG. 6 shows an exemplary audio and/or video conferencing system in which the television is used for conveying audio and/or video information to an individual and the remote controller with built-in microphone is used for conveying audio information from the individual, in accordance with an embodiment of the present invention. In this example, there are two remote locations 610 and 620 coupled to a head-end system 640 through various networks or the Internet 630. Also in communication with the head-end system 640 is a conference service provider 650 having a conferencing service server, service provider centers, equipment/maintenance service provider, and video conferencing connection service. Conferencing can be between two or more remote locations or between one or more remote locations and a service provider.

Among other things, the head-end system 640 configures and maintains the system configuration and routing of the information. The head-end system 640 also collects and distributes the equipment maintenance and repair information to the equipment maintenance provider. The head-end system 640 has the ability to connect to multiple service providers and communicate the appropriate information to and from them. The conference service provider 650, through the conferencing service server, configures and maintains the communication connections between locations. The conference service provider also has the capability to record the videoconferences at the head-end server or on other computers/servers.

In an exemplary embodiment of the invention, the interface at a remote location may receive audio signals from a remote controller and/or video signals from a video camera. The interface may digitize these signals. The interface transmits the digitized signals over the networks/Internet 630. The interface may also receive audio and/or video information from the networks/Internet 630. The interface may display video on the television. The interface may output audio on the television and/or the remote controller.

A remote servicing and monitoring system as described above may be used in a wide variety of remote servicing and monitoring applications. For example, a remote servicing and monitoring system may be used for such things as medical services and monitoring, educational services and monitoring, interactive advertising services, weather services, energy management services and monitoring, healthcare and fitness services, safety and security services, remote control, remote audio and/or video monitoring, and audio and/or video conferencing, to name but a few.

For medical services and monitoring, the input/output devices 220 at the remote location may include one or more medical devices that measure physiological parameters (such as, for example, blood pressure, body temperature, blood sugar) of the individual and provide parameter outputs to the interface 210. The interface 210 can process the parameters outputs locally and/or send the parameter outputs to one or more monitoring locations. Through the interface 210, medical information can be provided to the individual, such as, for example, reminder to take medications, first aid information, home remedy information, medicine information such as side-effects and precautions, surgery recovery information, or infectious/contagious disease management information. A video camera at the remote location may allow a medical services provider to visually inspect the individual, for example, to assess physical condition, to monitor the individual during a medical procedure (e.g., to make sure the individual performs a procedure correctly), or to confirm that the individual has completed a medical procedure (e.g., to confirm that the individual took the correct medication at the correct time by actually watching the individual take the medication).

In an exemplary embodiment of the invention, an individual at a remote location can tune to a predetermined television channel to receive medical information and/or communicate with a health care provider (e.g., doctor, nurse, family member). Thus, for example, if the individual does not feel well or has any other medical concerns (e.g., what medication to take), the individual tune to the predetermined television channel to receive medical information or request a videoconference with a medical professional or customer service provider. The medical information can be generated locally by the interface 210 or remotely from the monitoring facility. The medical information can be in multimedia format or simple test and picture format. The medical information can be “canned” or can be tailored to the individual, for example, based on physiological parameters generated by medical monitoring equipment, information provided by the individual (e.g., symptoms), or information contained in a profile (e.g., illness and medication information). The individual can also initiate a conference with a monitoring facility to speak with a health care provider. The health care provider is typically able to monitor the individual through the monitoring equipment, and may also be able to monitor the individual visually from a video camera at the remote location. The individual is typically able to see and/or hear the health care provider, which makes it easier for the health care provider to aid the individual (e.g., by walking the individual through a procedure).

Particularly where the interface 210 has Internet connectivity, the health care provider can be virtually anywhere in the world. This has many benefits for both the individual and the health care providers. For example, in order to save costs or for other reasons, health care providers could use a monitoring facility in another country (e.g., where doctor and nurse salaries are lower). Also, health care providers would not necessarily have to staff a particular monitoring facility 24 hours a day, but rather the individual can be made to communicate with different monitoring facilities at different times of the day in different parts of the world (e.g., a monitoring facility in the vicinity of the remote location during daytime, a monitoring facility overseas during nighttime). Individuals can be automatically directed to specialists if necessary. Furthermore, individuals can communicate with health care providers that satisfy language, religion, custom, and other concerns (e.g., a person from a foreign country can communicate with a health care provider in their native country). The profile stored at the interface 210 may include user preferences, such as, for example, preferred language, preferred gender of the health care provider, or religious restrictions (e.g., in some religions, a person cannot receive a blood transfusion). By storing the profile locally at the interface 210, the information in the profile can be maintained in secret and within the patient's/user's controls, and the profile can be applied to various transactions without revealing the contents of the profile. This provides for both security and privacy.

The servers at the remote location and/or the monitoring facility may allow for automatic monitoring, diagnosis, and limited treatment of an individual. For example, a server may receive physiological information from medical monitoring equipment as well as information from the individual (e.g., symptoms) and generate alerts based on some predetermined and/or configurable rules (e.g., generate alert if blood pressure too high or too low). The rules can be provided to the server by the individual and/or by the health care provider. The rules can define such things as “normal” and/or “abnormal” conditions for the patient, conditions under which an alert is to be generated, the type(s) of alerts to be generated, and to whom the alert is to be generated. For example, a doctor can prescribe a new medication for the individual and define a set of rules to, say, alert the doctor if there is no significant change in the patient's condition within some number of days and to immediately alert the doctor and a hospital if the patient's condition degrades beyond some degree.

The servers at the remote location and/or the monitoring facility may allow for providing automatic reminders to an individual, such as, for example, to take medications, initiate certain medical procedures, or take other actions. For example, information regarding an individual's medications can be entered into a server, for example, by the individual, a health care provider, or a pharmacist. This information may include such things as the type of medication, the dosage, and the frequency. The server can generate real-time reminders for the individual to take the medication. The reminders may be communicated to the patient/user by a signal that will flash an LED or sound the buzzer on the remote controller 240. Under some circumstances, the server may be able to monitor the individual to make sure the medication is taken (for example, by monitoring equipment used by the individual to take medications). The server may wait for a confirmation signal from the user that the medications have been taken.

Through patient monitoring, a health care provider can track such things as the amount of medication taken, the pattern/timing of medication, the effects of the medication, and/or other factors. Warnings can also be generated when medications or supplies are running low.

Educational services and monitoring can include such things as remote learning, remote testing, parent-teacher conferencing, or automated student reporting, to name but a few. Remote learning may involve unidirectional (e.g., providing lecture materials to an individual at a remote location), bi-directional (e.g., interactive classroom), and even multidirectional (e.g., study groups) communications. Remote testing may be enhanced through the use of a video camera at the remote location, for example, to confirm the identity of the test taker and to watch the test taker to make sure there is no cheating.

Interactive advertising services may be provided through the interface 210, which can filter advertisements intended for the individual (e.g., based on a profile stored locally at the interface 210) and allow the individual to dynamically select the types of advertisements to be shown (e.g., through the wireless remote controller). Advertising may be filtered based on local, regional, state, national, or global scale.

Weather services may be provided through the interface 210. For example, the interface 210 can obtain local, regional, state, national, or global weather information and present the weather information to the individual.

Energy management services and monitoring may involve such things as remote meter reading (e.g., gas, electric, water), remote monitoring of energy efficiency, providing energy cost information to the individual, and allowing the user to select energy management selection options, to name but a few. Remote control, diagnostics, and maintenance of utilities and related devices can be provided through the interface 210.

Thus, in certain embodiments of the invention, an individual in a first location is monitored by sensing at least one physiological parameter of the individual and providing at least one parameter output, providing information related to the at least one parameter output over a communication link to at least one monitoring facility in at least one location different from the first location (such as, for example, providing a first parameter output to a first monitoring facility and providing a second parameter output to a second monitoring facility), and using the communication link to permit video information derived from the first location to be conveyed to a monitoring facility and information including at least audio information derived from the monitoring facility to be conveyed to the first location. The communication link may also be used to permit video information derived from the monitoring facility to be conveyed to the first location. A server (e.g. at the monitoring facility or the remote location) may automatically determine that at least one parameter output reaches an alarm condition (such as, for example, determine that the at least one parameter output indicates a likelihood for a predetermined disease, e.g., SARS, smallpox, influenza, multi-drug resistant tuberculosis, congestive heart failure, asthma, diabetes, chronic obstructive pulmonary disease, heart attack, stroke, and seizure, to name but a few) and provide a parameter alarm indicating the alarm condition. The parameter alarm may be provided, for example, by placing a phone call, lacing a page to a paging device, communicating with a mobile phone, or communicating the proximity alarm over a digital communication network.

In another embodiment of the present invention, an individual in a first location is monitored by sensing at least one physiological parameter of the individual and providing at least one parameter output, providing information related to the at least one parameter output over a communication link to at least one monitoring facility in at least one location different from the first location (such as, for example, providing a first parameter output to a first monitoring facility and providing a second parameter output to a second monitoring facility), and using the communication link to permit information including at least audio information derived from the first location to be conveyed to a monitoring facility and video information derived from the monitoring facility to be conveyed to the first location. The communication link may also be used to permit video information derived from the first location to be conveyed to the monitoring facility. A server (e.g. at the monitoring facility or the remote location) may automatically determine that at least one parameter output reaches an alarm condition (such as, for example, determine that the at least one parameter output indicates a likelihood for a predetermined disease, e.g., SARS, smallpox, influenza, multi-drug resistant tuberculosis, congestive heart failure, asthma, diabetes, chronic obstructive pulmonary disease, heart attack, stroke, and seizure, to name but a few) and provide a parameter alarm indicating the alarm condition. The parameter alarm may be provided, for example, by placing a phone call, lacing a page to a paging device, communicating with a mobile phone, or communicating the proximity alarm over a digital communication network.

In another embodiment of the present invention, remote conferencing capability is provided to an individual at a first location. Remote conferencing typically involves using a television device at the first location, coupled through an interface to at least one communication link, to convey audio and/or video information from a second location to the individual and using a remote controller equipped with a microphone at the first location to direct operation of the interface and to provide an audio input to the interface. The audio information derived from the audio input is transmitted to the second location over the at least one communication link.

In order to use the television device at the first location to convey audio and/or video information to the individual, the audio and/or video information is typically received by the interface from a second location over the at least one communication link and transmitted by the interface to the television device, for example, on a predetermined television channel. The audio and/or video information may include such things as medical information (such as a reminder to take medication, first aid information, chronic disease management, home remedy information, medicine information such as side-effects and precautions, surgery recovery information, or infectious/contagious disease management information), educational information (such as lecture materials, remote testing, parent-teacher videoconferencing, or automated student reporting), advertising information (including local, regional, state, national, or global advertising information), weather information (including local, regional, state, national, or global weather information), or energy management information (such as a power meter reading, a water meter reading, a gas meter reading, energy efficiency information, energy cost information, or energy manufacturer selection options), to name but a few. At least some of the audio and/or video information conveyed to the individual may be selectable by the individual using the remote controller. At least some of the audio and/or video information may be conveyed to the individual based on a profile stored in the interface. The user's profile may include private information (such as, for example, diseases, medical conditions, medications, allergies), and this local storage and management of the user's profile helps to keep such information private while allowing it to be used to filter information presented to the user.

A video camera may be used to capture video information at the first location and transmitting the video information by the interface to a second location over the at least one communication link. The video information may be communicated to the interface in any of a variety of ways. For example, the video camera may be coupled directly to the interface or the video camera may be coupled or integral to the remote controller (in which case the video information is transmitted by the remote controller to the interface).

A remote conference may be initiated from either the first location or the second location or by a service provider. In one exemplary embodiment, the conference is initiated from the second location, for example, by the interface receiving an incoming signal from the second location over the at least one communication link indicating the start of a conference and activating the conference in response to the incoming signal. As part of activating the conference, the interface may transmit an alert signal to the remote controller in response to the incoming signal. The remote controller may, in turn, generate an alert to the individual in response to the alert signal in any of a variety of ways. For example, the remote controller may include an audio output device (such as a speaker or buzzer), in which case the remote controller may produce an audible sound from the audio output device to alert the individual. The remote controller may alternatively or additionally include a light emitting device (such as an LED), in which case the remote controller may activate the light emitting device to alert the individual. The remote controller may alternatively or additionally include a text display (such as an LCD display), in which case the remote controller may display a text message on the visual display to alert the individual.

In another exemplary embodiment, the conference is initiated from the first location using the remote controller. In this case, the remote controller typically transmits an alert signal to the interface indicating the start of a conference (for example, when a predetermined button on the remote controller is depressed by the individual), and the interface activates the conference in response to the alert signal. The interface may activate the conference in any of a variety of ways, such as, for example, transmitting an outgoing signal to the second location over the communication link in response to the alert signal.

As part of any conference, the interface may cause the television to be turned on (if not already on) and tuned to a predetermined television channel. An embodiment of the present invention may also support an “always on” form of conferencing in which audio and/or video information is streamed from the first location to the second location and/or from the second location to the first location and conveyed to the user over a predetermined television channel. If a user tunes to the appropriate channel, then the user receives the audio and/or video from the remote location. This can be done without notifying anyone at the remote location or with notifying the remote location, for example, by transmitting a signal over the at least one communication link. This would allow someone at one location, for example, so selectively monitor an individual at the remote location by simply tuning in to the appropriate channel and changing the channel (or turning off the television) to stop monitoring. In order to support this type of functionality, the interface may include a “block/add” capability in which a programmable filter blocks out the predetermined television channel (e.g., from cable, satellite, or antenna) and a programmable transmitter inserts a television signal on that television channel including the audio and/or video information from the remote location.

In embodiments of the present invention, the at least one communication link may include a persistent communication link allowing communication with the second location. The interface may include redundant communication interfaces to a plurality of communication links. The interface may alternatively or additionally include a first communication interface to a first communication link and a second communication interface to a second interface coupled to a second communication link. The at least one communication link may include communication over the Internet or a private intranet. The at least one communication link may include any of a variety of communication connections, such as cable modem, digital subscriber line (DSL), dial-up mode, or cellular modem.

In one exemplary embodiment of a remote healthcare system, the system utilizes low cost, low-bandwidth wireless modules for communication between medical monitoring devices and a hardware gateway device (referred to as a Television Interface Unit or TVI) that are installed in the home. Among other things, the TVI locally analyzes information received from the medical monitoring devices and selectively sends this information to one or more central computers (head-end servers) for review by health care providers. It is also capable of generating alarms and alerts for both the patient and the health care providers if data exceeds pre-set values or reaches a pre-configured state. The system preferably utilizes a broadband connection (cable broadband, DSL, cellular, or satellite) to permit the continuous transmission of vital sign data to the head-end server(s), to support the display of a wide variety of interactive health education and health status information to the patient, and to support high-quality videoconferencing.

The wireless modules are preferably installed at the medical monitoring devices. The wireless modules can use any of a variety of protocols, such as open band radio frequencies in open ISM band, Bluetooth, or IEEE 802.15.4/Zigbee. The modules generally have extremely low power requirements. The wireless modules are preferably integrated with the medical monitoring devices, for example, using serial communication protocols including either a TTL level signals or a RS232 port. The modules have a low power micro-controller that enables the modules to communicate over an in-home ‘master-slave’ wireless network.

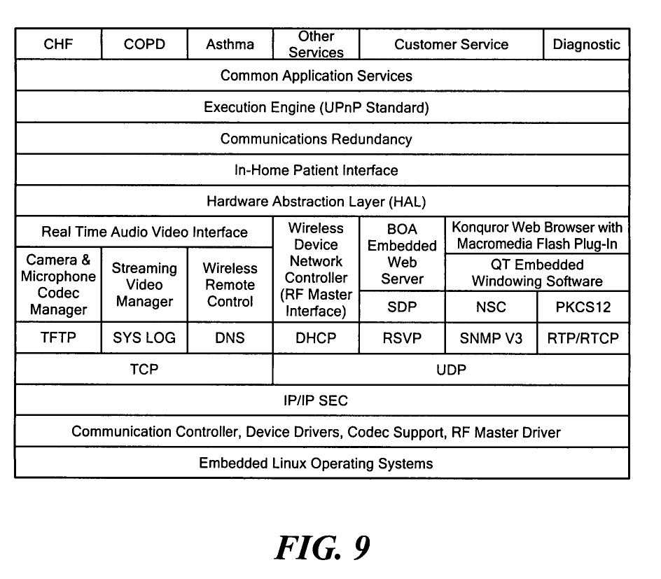

The TVI is the core component in the home. The TVI is a Linux™ based broadband gateway that includes complete networking functionality (including, among other networking functionalities, TCP/IP, DHCP, DNS, TFTP, RTP/RSTP, SNMP V3), an embedded web server (BOA), a web-browser, and remote communications tools. FIG. 9 shows an exemplary protocol stack for the TVI in accordance with an embodiment of the present invention.

In order for the TVI to connect to an external wide area network (WAN) or the Internet, the TVI can have an in-build broadband modem (e.g., cable modem or DSL) and/or an optional USB or Ethernet port to connect to an external modem. The TVI preferably also includes one or more wireless interfaces for wireless communication with various devices in the system. The TVI may have other wired and wireless interfaces, such as a power-line interface (e.g. LonWorks™ by Echelon) or a serial interface (e.g., RS422). The TVI may have multiple networks operating at the same time so that, for example, a printer can be connected over a wireless network or over an Ethernet LAN and medical devices can be connected on another wireless network. The TVI can be packaged in various forms, such as, for example, a “black box” that can be installed where the cable first comes into the home (e.g., basement or attic) so that the video can be viewed on any connected television set in the home, or as set-top box that sits close to or on top of a television set so that the video can be viewed just at that television set.

The video output of the TVI is sent over a television channel that is preferably uninterrupted by any other set-top or recording device in the home. The video output channel may be pre-configured during manufacturing or may be programmable. The video information sent to the television set(s) can be generated locally (e.g., by the TVI) and/or remotely (e.g., by the head-end server) and can include video information for such things as videoconferencing, multimedia education movies, charts, graphs, questionnaires, and medicine reminders, to name but a few.

In order to allow for video conferencing, the TVI preferably connects to an in-home video camera, for example, through a composite interface, a USB port, an Ethernet connection, or an IEEE 802.11b/g wireless interface. The videoconferencing is preferably IP based, which allows videoconferencing between various parties over the Internet so that, for example, a patient can videoconference with a doctor or nurse for healthcare, with a support engineer for device maintenance, with a care provider (e.g., son or daughter), with a pharmacist for medicine information, or with a customer service provider for general help.

The TVI preferably uses the latest H.264 video-compression technology that allows implementation of high quality video (up to 30 frames a second) over broadband. The TVI uses multiple advanced Video DSPs (Digital Signal Processors) to encode and decode the video signals to make it possible to send and receive the video signals over broadband

A wireless remote control serves as a navigation tool for the system. The remote control preferably includes the same wireless module as those used for the medical monitoring device in order to allow bi-directional communication with the TVI. The wireless remote control preferably includes various buttons that allow for navigation of television displays and other control functions. The wireless remote control preferably includes various output devices, such as a LEDs and a buzzer, that can be controlled by the TVI for signaling to the user. The remote control preferably includes an in-built microphone that allows for audio input for interactive multimedia communications.

A Pocket PC can also be used by a patient to interface with the system. The Pocket PC offers a simple and creative way for the user to enter and view information in text. The Pocket PC will communicate with TVI within the home.

The wireless network protocol allows a master RF module to periodically poll in-home devices with RF modules. The RF master modules also facilitate the peer-peer communications between the in-home devices. Multiple master and redundant master RF modules can exist on the network. The protocol is designed for small devices and after initial configuration, requires no user interface or maintenance. The wireless modules are designed to be interchangeable, and other wireless technologies can be used (e.g., Bluetooth and IEEE 802.15.4/Zigbee). In this embodiment, the TVI is designed to include up to four RF master modules at a time so as to allow up to four different in-home device networks to coexist at a time.

The head-end server acts as the collection, application, and distribution server for the system. In this embodiment, the head-end server uses JBOSS and SQL database. The head-end server preferably has a global static IP address and a ‘private’ encryption key that is embedded during manufacturing. The head-end server uses a robust database to organize data and execute various applications periodically and in real-time to analyze the collected information. The applications take input from a number of service providers and generate reports, alarms, and alerts based on the available information. The head-end server also allows service providers and users to configure the delivery methods for the reports, alarms, and alerts. The head-end server can also store a number of multimedia education movies that can be online downloaded to any TVI based on the configuration. This allows the service providers to enable and make available to the user very specific information that corresponds to their conditions and needs. The information delivered this way can include such things as step-by-step processes about how to use a medical device and information about how to control certain chronic symptoms of the disease.

The collection server component of the head-end server communicates with the TVIs over the Internet using authentication and 128-bit encryption and SSL. Communication between the collection server and the TVIs is based on an XML-based protocol over IP. The collection server includes databases to store all the information collected from remote TVIs. The architecture is scalable to allow communication and data collection of information from thousands of remote TVIs. This includes creation of database records for each TVI and establishment of the number of retries, frequency of polling, and data bandwidth for each TVI. The collection server also includes a web-based interface to configure the collection server and manage the information stored in the databases. The collection server also includes a backup for the database.

The application software component of the head-end server takes the information collected from the remote patients and analyzes it to generate reports, alarms, notifications, and recommendations. It creates the web pages for the individual user that can be viewed remotely on the television. It allows the service providers to view information, such as trends and reports for individual patients, and enter comments, data, prognosis, recommendation, and other information that can be viewed by the patient remotely on the television. The application server has different levels of security including password authentication, encryption of messages, and application level encryptions to ensure security and privacy of patient data and information. A 128-bit encryption scheme is used for the patient information to provide privacy. The application server can help automate the analysis and diagnostic process.

The distribution server component of the head-end server distributes appropriate information to the appropriate service providers. The architecture of the distribution server supports multiple service provider interfaces, such as an interface to a medical service professional like a nurse or a doctor to provide patient's vital sign data or an interface to a public health provider to send interactive educational information to the patients. The distribution server also manages the delivery and recording of alarm and alerts. It can send an automated alarm or report via e-mail, telephone, cell phone, pager or Internet. Communications between the distribution server and the service providers are based on XML-based protocols over IP. The system administrator uses a web-based configuration tool to allow service providers to access information in the head-end server. Service provider logins are protected using authentication and password protection.

The basic web-based service provider interface includes graphical interfaces for the display and organization of collected data for the healthcare service providers. It also includes easy-to-use configuration tools for viewing the information in the form of reports and charts with alarms. It also includes a notification system for notifying healthcare providers via e-mail, cell-phones, pager, and other means. It also allows the healthcare service providers to login and provide disease management services to the patients, including, for examples scheduling videoconferences, reviewing vial signs, adding or modifying a questionnaire for the patients, and enabling patient viewing of customized disease management video, among others. It also allows small home healthcare agencies to provide services via web access without requiring them to have in IT staff to implement and maintain the server to manage the information.

In a basic tele-healthcare system, data can be collected in real time from such things as a blood pressure and pulse measuring instrument, a weight scale, a pulse oximeter, a thermometer, and/or a spirometer. Dynamic real-time analysis can be performed on the data. Critical alarms and reports can be generated. The alarm notification can be sent based on configured parameters via e-mail. Reports and notifications can be viewed by specialists via secure Internet access.

The ability to send video output to the television set allows for a multimedia-based, interactive patient interface that can be managed with the remote control. This interface allows the patients to view their health status, vital signs, and health trends over the television set. In addition, the healthcare provider can interact with patients, for example, by videoconferencing, by having questions displayed on the television set and having the patient answer the questions using the remote control, by displaying medicine reminders on the television set, and by providing multimedia education information on the television set, to name but a few.

The system is preferably capable of downloading different applications to the TVI. This customizability of the IRG through downloaded toolkits will allow each TVI to be customized and optimized for a particular monitoring task. For example, the applications to manage congestive heart failure patients will generally be different from the applications for chronic obstructive pulmonary disease patients, including use of different sets of devices, different care algorithms, and different types of alarms and alerts.

Because the TVI generates a video output signal to the television set(s), the TVI can encode and transmit the same video signal (or portions thereof) to the head-end server to be viewed by a service provider. In this way, the service provider can see exactly what the patient sees. A remote signaling system, such as a remote “whiteboard” system, can be used to allow the service provider to manipulate and overwrite the video display being viewed by the patient. Thus, for example, the service provider can point to or highlight specific information on the video display or add typed or handwritten notes to the video display.

Certain embodiments of the present invention provide for remote monitoring of an individual in situations where it is necessary or desirable to monitor not only physiological (e.g., medical) information, but also the physical presence of the individual within some proximity, for example, due to detention of the individual. Certain embodiments of the present invention are envisioned for remote medical monitoring in disease quarantine situations (e.g., SARS, smallpox, influenza, multi-drug resistant tuberculosis, congestive heart failure, asthma, diabetes, chronic obstructive pulmonary disease, heart attack, stroke, and seizure, to name but a few), although embodiments can certainly be used for remote monitoring in many other situations in which it is necessary or desirable to monitor both physiological information and proximity, including, but in no way limited to, remote monitoring of prisoners, individuals under house arrest, mental health patients, nursing home patients, Alzheimer patients, and individuals who are a flight risk, to name but a few.

Various aspects of the present invention are described herein with reference to remote medical monitoring in a disease quarantine situation, although it will be apparent that these aspects apply generally to other remote monitoring situations. A typical embodiment of the present invention allows health care workers to remotely monitor the medical condition of an individual. Among other things, this allows the health care workers to monitor individuals who are quarantined at different locations and also allows the health care workers to monitor the individuals without repeatedly or unnecessarily exposing health care workers to the quarantined individuals. A typical embodiment of the present invention also provides for monitoring the proximity of the various quarantined individuals to prevent, or at least detect, a quarantined individual leaving the quarantine location or removing, disabling, or otherwise circumventing proximity monitoring equipment.

In an exemplary embodiment of the present invention, the remote medical monitoring system includes two sets of hardware/software components, one that is typically used in the quarantine location and one that is typically used in a monitoring facility remote from the quarantine location (referred to hereinafter as the “head-end”). The equipment at the quarantine location and the head-end equipment are in communication over a communication network, and typically communication by exchanging packets of digital information.

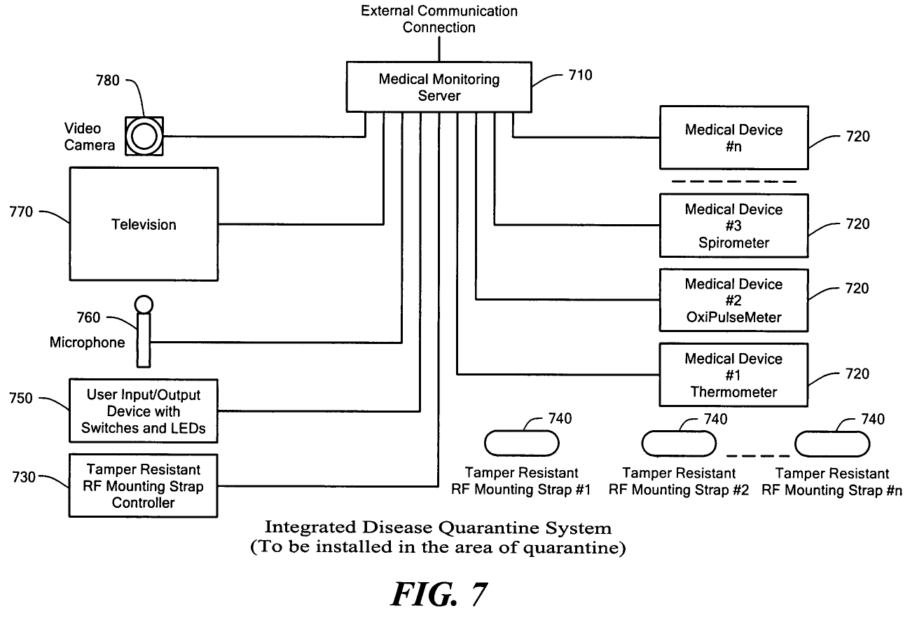

FIG. 7 shows an exemplary set of hardware/software components designed to be used in the quarantine location in accordance with an embodiment of the present invention. This set of hardware/software components typically enables such things as the collection, storage, analysis, and display of vital sign data gathered from the quarantined individual(s), proximity detection, and videoconferencing between the quarantined individual(s) and the health care workers, among other things. This set of hardware/software components typically includes a medical monitoring server 710 that is in communication with various monitoring devices 720 (medical devices 1 through n) that monitor physiological parameters (e.g., vital signs), an electronic proximity detector 730 (e.g., a tamper resistant RF mounting strap controller) that determines proximity based on signals generated by proximity input devices 740 (e.g., tamper resistant RF mounting straps 1 through n that are worn by individuals), and a videoconferencing platform including a cable-ready television 770, a video camera 780, a microphone 760, and a user input/output device 750 with switches and LEDs. The medical monitoring server 710 receives signals from the various vital sign monitors 720 as well as from the electronic proximity detector 730. The medical monitoring server 710 also interacts with the videoconferencing platform 750-780 to send and/or receive audio and/or video information. The medical monitoring server 710 is coupled to a communication network through an external communication connection, for example, using an internal modem (e.g., POTS, cable, DSL, or cellular modem) or via connection (e.g., via Ethernet, serial, or USB connection) to an external modem. The present invention is in no way limited to any particular communication networks or technologies.

The monitoring devices 720 collect physiological parameters from the quarantined individual(s) and transmit this information to the medical monitoring server 710. A wide range of monitoring devices can be used. FIG. 7 depicts a thermometer, an OxiPulseMeter, and a Spirometer. The monitoring devices 720 can be in communication with the medical monitoring server 710 through wired and/or wireless communication links.

The electronic proximity detector 730 (in this example, the mounting strap controller) detects proximity of the quarantined individual(s) and generates signals to the medical monitoring server 710 including proximity information. In this example, the mounting strap controller 730 typically picks up signals generated wirelessly by the mounting straps 740 and sends signals to the medical monitoring server 710 indicating proximity information. If a quarantined individual moves a sufficient distance from the mounting strap controller 730, then the mounting strap controller 730 will no longer receive a signal from the mounting strap 740 worn by that individual, and this condition will preferably be detected by the medical monitoring server 710 through appropriate signals received from the mounting strap controller 730. The mounting strap controller 730 is preferably also able to detect that a mounting strap 740 has been removed or otherwise disabled by the wearer, and this condition will preferably be detected by the medical monitoring server 710 through appropriate signals received from the mounting strap controller 730.

The medical monitoring server 710 acts as a data gateway. Among other things, the medical monitoring server 710 collects certain types of information from the monitoring devices 720, the electronic proximity detector 730, and the videoconferencing platform 750-780 and transmits this information to one or more remote monitoring facilities over the communication network. The medical monitoring server 710 can also receive audio and/or video information from one or more remote monitoring facilities and present the audio/video information to the individual(s) using the videoconferencing platform 750-780 (e.g., playing audio and/or video information through the television set). This system therefore has the capability of supporting videoconferencing over the television set 770. The medical monitoring server 710 is capable of utilizing multiple communication technologies, such as cable internet, DSL, and cellular.

More specifically, the medical monitoring server 710 receives physiological parameters from the monitoring devices 720, converts the physiological parameters into digital data streams, and transmits this information over the communication network to a remote monitoring facility. The medical monitoring server also receives proximity information from the electronic proximity monitor 730 and sends proximity information over the communication network to a remote monitoring facility. The medical monitoring server 710 may also receive video signals from the video camera 780 and/or audio signals from the microphone 760, convert the audio/video information into digital data streams as necessary, and transmit this information over the communication network to a remote monitoring facility.

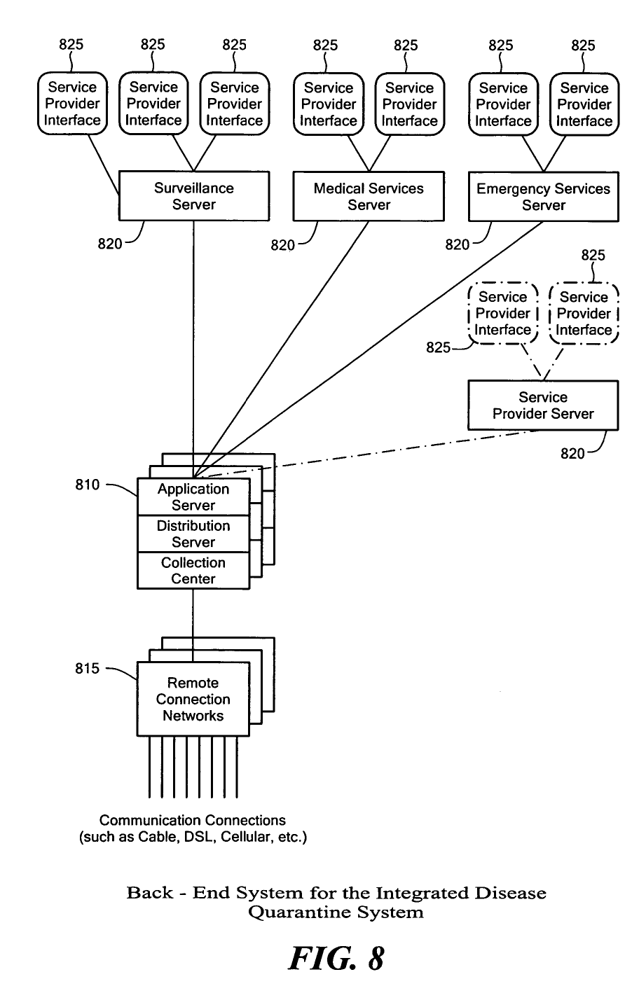

FIG. 8 shows an exemplary set of hardware/software components designed to be used in a monitoring facility remote from the quarantine location in accordance with an embodiment of the present invention. This set of hardware/software components is designed to assist in the monitoring of the quarantined individual(s) at the head-end. Among other things, this set of hardware/software components includes a head-end server 810 (application/distribution/collection) that connects to multiple service provider servers 820 as well as to the quarantine locations through one or more remote connection networks 815. Each server provider server 820 includes one or more service provider interfaces 825 for access by a service provider. The service provider interfaces 825 can use any of a variety of communication technologies, such as a computer with a web browser, a cellular phone, a pager, or other type of communication and reporting technologies.

The head-end server 810 receives physiological parameters, proximity information, and possibly video and/or audio information from one or more remote medical monitoring servers via the remote connection network(s) 815. The head-end server 810 runs a set of commercial and proprietary software that translates the digital signals into interpretable information. This information is preferably displayed using graphical user interface software that transmits both numerical data from the monitoring devices, as well as graphically-displayed trend data. The video signals are translated to broadcast on connected personal computers or other devices. Software running on the head-end server 810 includes programs that can automatically determine when an alarm condition exists and generate an appropriate alarm, for example, when a physiological parameter is out of a predetermined range (e.g., blood pressure too high), when one or more physiological parameters indicate the likelihood of a predetermined disease or condition (e.g., SARS, smallpox, influenza, multi-drug resistant tuberculosis, congestive heart failure, asthma, diabetes, chronic obstructive pulmonary disease, heart attack, stroke, and seizure, to name but a few), or when a quarantined individual is outside of a predetermined proximity (e.g., either because the individual is outside the range of the electronic proximity detector or the individual removed or otherwise disabled the proximity device).

The head-end server 810 information can be monitored by non-medical personnel (by logging in to the system using password authentications) who are responsible for the overall health of the subject and are assuring their physical presence in the quarantine area. The information can also be monitored by medical personnel, who can interpret the physiological parameters in terms of needed medical intervention. The medical personnel could also use the system to conduct simple physical examinations, and to monitor that the quarantined subject is properly performing the vital sign measurements, or to monitor the taking of medications.

It should be noted that there can be multiple monitoring facilities, and the monitoring facilities can be configured by function. For example, one monitoring facility (e.g., a health care facility) can be used to receive and process physiological information from the medical monitoring server, while another monitoring facility (e.g., a security office) can be used to receive and process proximity information from the medical monitoring server.

The alarm can be generated to one or more third parties that can be at or away from the monitoring facility. For example, the head-end server 810 may generate an alarm by placing a phone call, placing a page to a paging device, communicating with a mobile phone, and communicating over a digital communication network (such as the Internet).

In one scenario, certain people may be quarantined at an insecure location (e.g., home or hotel) due to possible exposure to a disease and other people actually suspected of having the disease may be quarantined at a more secure location. In such a scenario, it becomes important to identify those individuals quarantined at their homes who begin to show signs of having the disease so that they can be moved to the secure facility. Embodiments of the present invention enable this by automatically determining when physiological parameters indicate the likelihood of a predetermined disease and generating an alarm. The alarm may indicate which individual(s) to move to the secure location.

In another scenario, a person may be quarantined at a secure facility. In such a scenario, it may be important to notify both a health care worker and security personnel if the person leaves the quarantine location. Embodiments of the present invention enable this by automatically detecting a proximity alarm condition and generating alarms to both health care workers and security personnel.

Thus, embodiments of the present invention may involve sensing a physiological parameter of the individual, a proximity of the individual, and/or a location-specific parameter, providing a parameter output to the interface, and providing information related to the parameter output by the interface over a communication link to at least one monitoring facility in at least one location different from the first location. Information may be conveyed to the individual through a television device based on the parameter output.

Monitoring an individual who is subject to detention in a first location may involve sensing at least one physiological parameter of the individual and providing a parameter output, sensing the proximity of the individual and providing a proximity output, and providing information related to the parameter output and the proximity output over a communication link to at least one monitoring facility in at least one location different from the first location.

In a related embodiment, such monitoring may also involve using the communication link to permit video information derived from the first location to be conveyed to a monitoring facility and information including at least audio information derived from the monitoring facility to be conveyed to the first location. In a further related embodiment, using the communication link may further involve using the communication link to permit video information derived from the monitoring facility to be conveyed to the first location.

In another related embodiment of the invention, a server (e.g., at a monitoring facility or at a remote location) may automatically determine that the proximity output reaches an alarm condition and provide a proximity alarm indicating the alarm condition. In a further related embodiment, determining automatically may involve determining that the individual is outside the range of a proximity sensor. In the alternative, determining automatically may involve determining that the individual has removed a proximity device. In another further related embodiment, providing a proximity alarm may involve at least one of automatically placing a phone call, placing a page to a paging device, communicating with a mobile phone, and communicating the proximity alarm over a digital communication network.

In still another related embodiment of the invention, information related to the parameter output may be provided to at least one parameter monitoring facility and information related to the proximity output may be provided to at least one proximity monitoring facility.

It should be noted that the remote communication networks 120 can include multiple communication technologies. At least one of the communication links between a remote location and a head-end server is an “always on” connection. Multiple remote locations, head-end servers, monitoring locations, and others may be in communication over one or more communication networks, allowing, among other things, conferencing, monitoring, servicing, and other functions between two or more locations.

The use of “always on” communications can allow for streaming of information (e.g., video and/or audio information) amongst the various locations. Thus, for example, audio and/or video information from a first location A can be streamed to a second location B, and audio and/or video information from location B can be streamed to location A. A person at location A can tune to a predetermined television channel to monitor location B, and a person at location B can tune to a predetermined television channel to monitor location A. Such tuning can be used for passive monitoring (e.g., without notifying the person at the monitored location) or for initiating a two-way or multi-way conference (e.g., by notifying the person at the monitored location). A person at either location can typically “turn off” the ability of the other location to monitor, for example, by turning the interface off, although the interface may be remotely controllable to turn on the interface and thus enable monitoring. This feature could be used, for example, for someone to remotely monitor a family member who may inadvertently shut off the interface (e.g., an Alzheimer's patient).

The use of “always on” communications is also advantageous because it enables near-instantaneous communications, without delays for dynamically establishing communication (e.g., through a dial-up modem). Thus, for example, medical emergencies can be quickly detected and communicated to a remote monitoring facility (or elsewhere), so help can be provided in a timely manner. This can be particularly important in life-threatening situations, such as heart attack, stroke, choking, drowning, or diabetic shock, where medical help must be administered quickly.

Thus, embodiments of the present invention can be used in various scenarios to provide remote monitoring and servicing. For example, medical patients or others situated at one location (e.g., home, hospital, clinic, nursing home, assisted living facility, or other location) may be monitored and serviced by one or more care providers (e.g., doctor, nurse, pharmacist, family member, guardian, expert/consultant) situated at another location (e.g., home, hospital, clinic, nursing home, assisted living facility, or other location). Medical and other monitoring/servicing equipment (e.g., TVI, television, camera, microphone) can be located in publicly available places (e.g., pharmacy, supermarket, mall, health clinic, mobile health center), for example, as a kiosk, to provide the described types of monitoring and servicing, by appointment or on-demand. Communal monitoring and servicing can be provided (e.g., in a hospital, nursing home, assisted living facility, or other resident or non-resident facility) by having a single television shared by multiple patients for videoconferencing or distributing various types of information (e.g., education, medicine, exercise) with groups of patients. Individual and communal systems can also be used to provide pre-operative and post-operative information both in pre-recorded form and through videoconferencing. Various types of reminders (e.g., when to take medication, when to stop eating in preparation for a medicine or procedure, when to begin or end a procedure) and related information (e.g., description of medicine, dosing, side effects, what to do if a dose was missed, when to call a nurse or doctor, conditions for taking the medicine such as must be on a full stomach or must be on an empty stomach, drug interactions, things to avoid when on the medication, possible alternative medications or treatments including generic or over-the-counter substitute) can be provided through the television and/or the remote controller. Information gathered by the system (e.g., from the service provider, patient, monitoring devices, camera, microphone, or other input device) can be recorded for evidentiary or other purposes (e.g., as proof that the patient was given certain information, as proof that the patient gave informed consent to a procedure). Such recording can be performed by the interface devices, by the head-end server, and/or by the service provider servers.

Embodiments of the present invention are particularly advantageous for sleep disorder studies and other scenarios (e.g., agoraphobics) in which there is an actual or perceived benefit to monitoring/servicing the patient at home. With regard to sleep disorder studies, the patient is more likely to exhibit normal sleep patterns at home as compared to an in-patient facility where the patient is unfamiliar with the surrounding and may actually have problems falling or remaining asleep.

Embodiments of the present invention can provide other advantages, such as fast and automated diagnosis of certain conditions, such as heart attack or stroke. These conditions generally require immediate attention, and the local application in the TVI can help diagnose the condition and automatically contact the appropriate service provider.

Embodiments of the present invention also provide for direct and targeted advertising and marketing. Information can be provided to the user in audio and/or video form, and that information can be targeted specifically for the user based on various factors (e.g., medical condition, medicine prescription). The information can include advertisements for such things as specific service providers (e.g., hospitals, doctors, physical therapy centers), consumer products, home health care services, home delivery services, home equipment maintenance services, pharmaceutical products, automotive repair services, local community information, home improvement services, baby sitting and daycare services, educational/tutoring services, fitness programs, and weight loss programs, to name but a few.

In certain embodiments of the present invention, it may be necessary or desirable to be able to positively identify users who are being monitored or serviced, for example, to ensure that physiological information is being received from the correct person. Therefore, embodiments of the present invention may include user authentication devices, such as biometric sensors, for authenticating users. The authentication devices are typically located at the user location, and the gateway may perform authentication locally and/or forward authentication information to the head-end server and/or service provider servers.

In preferred embodiments of the present invention, the various input/output devices, including physiological monitoring devices, proximity monitoring devices, biometric authentication devices, and videoconferencing devices, are monitored regularly and automatically, for example, by the head-end server, the interface device, and/or the service provider server(s). Status information can be obtained. Various functions, such as diagnostics, calibration, and operation, can be performed. Alarms, reports, and other status information can be generated. The status information can be viewed by the service providers or others. Service and other requests can be generated for such things as error reporting, service requests, and ordering replacement components, for example, to the device manufacturer, a sales representative, or a service company. For example, a request can be automatically generated to order replacement cartridges for a glucose meter when it is determined that replacement will soon be needed.

It should be noted that terms such as “server” and “gateway” are used herein to describe various devices that may be used in a communication system, and should not be construed to limit the present invention to any particular type or types of communication devices. Thus, a communication device may include, without limitation, a bridge, router, bridge-router (brouter), switch, node, or other communication device.

It should also be noted that the term “packet” is used herein to describe a communication message that may be used by a communication device (e.g., created, transmitted, received, stored, or processed by the communication device) or conveyed by a communication medium, and should not be construed to limit the present invention to any particular communication message type, communication message format, or communication protocol. Thus, a communication message may include, without limitation, a frame, packet, datagram, user datagram, cell, or other type of communication message.

It should also be noted that the logic flow diagrams are used herein to demonstrate various aspects of the invention, and should not be construed to limit the present invention to any particular logic flow or logic implementation. The described logic may be partitioned into different logic blocks (e.g., programs, modules, functions, or subroutines) without changing the overall results or otherwise departing from the true scope of the invention. Often times, logic elements may be added, modified, omitted, performed in a different order, or implemented using different logic constructs (e.g., logic gates, looping primitives, conditional logic, and other logic constructs) without changing the overall results or otherwise departing from the true scope of the invention.

The present invention may be embodied in many different forms, including, but in no way limited to, computer program logic for use with a processor (e.g., a microprocessor, microcontroller, digital signal processor, or general purpose computer), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof. In a typical embodiment of the present invention, predominantly all of the medical monitoring server logic and the head-end server logic is implemented as a set of computer program instructions that is converted into a computer executable form, stored as such in a computer readable medium, and executed by a microprocessor under the control of an operating system.

Computer program logic implementing all or part of the functionality previously described herein may be embodied in various forms, including, but in no way limited to, a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies (e.g., Bluetooth), networking technologies, and internetworking technologies. The computer program may be distributed in any form as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality previously described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL).

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

The present invention may be embodied in other specific forms without departing from the true scope of the invention. The described embodiments are to be considered in all respects only as illustrative and not restrictive.

1. A method of monitoring an individual in a first location, the method comprising:
sensing at least one physiological parameter of the individual and providing at least one parameter output;
providing information related to the at least one parameter output over a communication link to at least one monitoring facility in at least one location different from the first location; and
using the communication link to permit video information derived from the first location to be conveyed to the at least one monitoring facility and information including at least audio information derived from the at least one monitoring facility to be conveyed to the first location.

2. A method according to claim 1, further comprising:
using the communication link to permit video information derived from the at least one monitoring facility to be conveyed to the first location.

3. A method according to claim 1, further comprising:
using the communication link to permit audio information derived from the first location to be conveyed to the at least one monitoring facility.

4. A method according to claim 1, wherein different parameter outputs are provided to different monitoring facilities.

5. A method according to claim 1, further comprising:
determining automatically that at least one parameter output reaches an alarm condition; and
providing a parameter alarm indicating the alarm condition.

6. A method according to claim 5, wherein providing a parameter alarm comprises at least one of:
placing a phone call;
placing a page to a paging device;
communicating with a mobile phone; and
communicating the parameter alarm over a digital communication network to a computer or other electronic device.

7. A method according to claim 5, wherein the alarm condition is determined by a server at the at least one monitoring facility.

8. A method according to claim 5, wherein the alarm condition is determined by a server at the first location and where the parameter alarm is provided to at least one of the at least one monitoring facility and the individual.

9. A method according to claim 5, wherein the alarm condition indicates the likelihood for a predetermined disease.

10. A method according to claim 9, where the predetermined disease is one of:
SARS;
smallpox;
influenza;
multi-drug resistant tuberculosis;
congestive heart failure;
asthma;
diabetes;
chronic obstructive pulmonary disease;
heart attack;
stroke; and
seizure.

11. A method according to claim 2, further comprising:
outputting a video signal to a video monitor at the first location, the video signal generated from at least one of:
the video information conveyed from the at least one monitoring facility; and
video information stored locally at the first location.

12. A method according to claim 11, wherein the video signal is output on a predetermined television channel.

13. A method according to claim 11, further comprising:
using the communication link to convey a representation of the same video signal to the at least one monitoring facility so that the same video signal can be viewed at both the first location and the at least one monitoring facility.

14. A method according to claim 1, further comprising:
outputting an audio signal to an audio output device at the first location, the audio signal generated from at least one of:
the audio information conveyed from the at least one monitoring facility; and
audio information stored locally at the first location.

15. A method according to claim 14, wherein the audio output device is a wireless remote controller with an in-built speaker, and wherein the audio signal is conveyed over a wireless communication link to the wireless remote controller.

16. A method according to claim 14, wherein the audio output device is a television set, and wherein the audio signal is conveyed as part of a television signal transmitted to the television set.

17. A method according to claim 3, wherein the audio information derived from the first location is input using a wireless remote controller with an in-built microphone.

18. A method according to claim 1, wherein the first location includes at least one physiological sensing device, and wherein sensing the at least one physiological parameter includes receiving signals from the at least one physiological sensing device.

19. A method according to claim 18, wherein the signals are received from the at least one physiological sensing device over a wireless communication system.

20. A method according to claim 1, further comprising:
sensing a proximity of the individual and providing at least one proximity output; and
providing information related to the at least one proximity output over the communication link to the at least one monitoring facility.

21. A method according to claim 11, further comprising:
determining automatically that at least one proximity output reaches an alarm condition; and
providing a proximity alarm indicating the alarm condition.

22. A method according to claim 21, wherein providing a proximity alarm comprises at least one of:
placing a phone call;
placing a page to a paging device;
communicating with a mobile phone; and
communicating the proximity alarm over a digital communication network to a computer or other electronic device.

23. A method according to claim 21, wherein the alarm condition is determined by a server at the at least one monitoring facility.

24. A method according to claim 21, wherein the alarm condition is determined by a server at the first location and where the proximity alarm is provided to at least one of the at least one monitoring facility and the individual.

25. A method according to claim 21, wherein the alarm condition indicates the likelihood that the individual is outside the range of a proximity sensor.

26. A method according to claim 21, wherein the alarm condition indicates that the individual has removed a proximity device.

27. A method according to claim 20, wherein the information related to the at least one parameter output and the information related to the at least one proximity output are provided to different monitoring facilities.

28. A method according to claim 1, wherein the communication link is a broadband communication link.

29. A method according to claim 1, wherein the communication link traverses the Internet.

30. Apparatus for remotely monitoring and servicing an individual at a first location, the apparatus comprising:
a first communication interface for communicating with at least one remotely located monitoring facility over a first communication network;
at least one second communication interface for communicating with a plurality of local input/output devices including at least one physiological input device that senses at least one physiological parameter of the individual and provides at least one corresponding physiological input signal, a video input device that provides video input signals, and an audio output device that receives audio output signals; and
a controller coupled to the first communication interface for communication with the at least one monitoring facility and to the at least one second communication interface for communication with the plurality of local input/output devices, wherein the controller selectively conveys information related to the at least one physiological input signal and video information derived from the video input signals to the at least one monitoring facility using the first communication interface, and wherein the controller selectively conveys audio information including audio information received from the at least one monitoring facility as audio output signals to the audio output device.

31. Apparatus according to claim 30, wherein the plurality of local input/output devices includes a video output device that receives video output signals, and wherein the controller selectively conveys video information including video information received from the at least one monitoring facility as video output signals to the video output device.

32. Apparatus according to claim 30, wherein the plurality of local input/output devices includes an audio input device that provides audio input signals, and wherein the controller selectively conveys audio information derived from the audio input signals to the at least one monitoring facility using the first communication interface.

33. Apparatus according to claim 30, wherein the controller selectively conveys parameter signals from different physiological input devices to different monitoring facilities.

34. Apparatus according to claim 30, wherein the controller selectively processes the at least one physiological input signal and provides a parameter alarm upon determining that at least one physiological input signal reaches a predetermined alarm condition.

35. Apparatus according to claim 34, wherein the controller provides the parameter alarm in the form of at least one of:
a phone call;
a page to a paging device;
a communication with a mobile phone; and
a communication over a digital communication network.

36. Apparatus according to claim 34, wherein the alarm condition indicates the likelihood for a predetermined medical condition.

37. Apparatus according to claim 36, where the predetermined medical condition is one of:
SARS;
smallpox;
influenza;
multi-drug resistant tuberculosis;
congestive heart failure;
asthma;
diabetes;
chronic obstructive pulmonary disease;
heart attack;
stroke; and
seizure.

38. Apparatus according to claim 31, wherein the at least one second communication interface includes a television output interface for communication with the video output device, and wherein the controller provides video output signals on a predetermined television channel through the television output interface.

39. Apparatus according to claim 38, further comprising a television input interface for receiving television input signals on a plurality of television channels, wherein the controller blocks television input signals on the predetermined television channel and inserts the video output signals on the same television channel through the television output interface.

40. Apparatus according to claim 39, wherein the controller passes television input signals from at least one other television channel through to the television output interface.

41. Apparatus according to claim 31, wherein the controller selectively conveys a representation of the video output signals to the at least one monitoring facility using the first communication interface so that the same video content can be viewed at both the first location and the at least one monitoring facility.

42. Apparatus according to claim 31, wherein the video information further includes video information stored in a local video storage.

43. Apparatus according to claim 30, wherein the audio information further includes audio information stored in a local audio storage.

44. Apparatus according to claim 30, wherein the at least one second communication interface includes a wireless communication interface for communication with a wireless remote controller having an in-built speaker, and wherein the controller conveys the audio information as audio output signals to the wireless remote controller over the wireless communication interface.

45. Apparatus according to claim 30, wherein the at least one second communication interface includes a television output interface for communication with a television, and wherein the controller conveys the audio information as audio output signals to the television on a predetermined television channel through the television output interface.

46. Apparatus according to claim 42, wherein the at least one second communication interface includes a wireless communication interface for communication with a wireless remote controller having an in-built microphone, and wherein the controller receives the audio input signals from the wireless remote controller over the wireless communication interface.

47. Apparatus according to claim 30, wherein the at least one second communication interface includes a wireless communication interface for communication with the at least one physiological input device, and wherein the controller receives physiological input signals from the at least one physiological input device over the wireless communication interface.

48. Apparatus according to claim 30, wherein the plurality of local input/output devices includes at least one proximity sensing device that senses a proximity of the individual and provides at least one proximity input signal, and wherein the controller selectively conveys information related to the at least one proximity input signal to the at least one monitoring facility using the first communication interface.

49. Apparatus according to claim 48, wherein the controller processes the at least one proximity input signal and provides a proximity alarm upon determining that at least one proximity input signal reaches a predetermined alarm condition.

50. Apparatus according to claim 49, wherein the controller provides the proximity alarm in the form of at least one of:
a phone call;
a page to a paging device;
a communication with a mobile phone; and
a communication over a digital communication network.

51. Apparatus according to claim 49, wherein the alarm condition indicates the likelihood that the individual is outside a predetermined range of the proximity sensing device.

52. Apparatus according to claim 49, wherein the alarm condition indicates that the individual has removed the proximity sensing device.

53. Apparatus according to claim 48, wherein the controller conveys the information related to the at least one physiological input signal and the information related to the at least one proximity input signal to different monitoring facilities.

54. Apparatus according to claim 30, wherein the controller selectively permits remote control of the plurality of local input/output devices by the at least one monitoring facility using the first communication interface.

55. Apparatus according to claim 30, wherein the controller establishes a secure communication connection with the at least one monitoring facility for secure communication with the at least one monitoring facility.

56. Apparatus according to claim 30, wherein the controller includes a web browser for accessing web pages over the first communication interface, and wherein the controller selectively conveys video information including representations of the web pages as video output signals to the video output device.

57. Apparatus according to claim 30, wherein the controller includes a web server for generating web pages.

58. Apparatus according to claim 34, wherein the controller uses the first communication interface to obtain user-specific applications for processing the at least one physiological input signal.

59. Apparatus according to claim 58, wherein the user-specific application processes the at least one physiological input signal based on a specific condition of the individual.

60. Apparatus according to claim 31, wherein the at least one second communication interface includes a video output interface for communication with a video monitor device.

61. Apparatus according to claim 60, wherein the video output interface is a VGA interface.

62. Apparatus according to claim 28, wherein the first communication interface is a broadband interface.

63. Apparatus according to claim 28, wherein the first communication interface is an interface to the Internet.

64. A system for remote monitoring and servicing, the system comprising:
a head-end server;
at least one service provider server in communication with the head-end server, each service provider server located at a service provider location;
at least one interface device in communication with the head-end server, each interface device located at a remote monitoring and servicing location having a plurality of local input/output devices in communication with the interface device, each plurality of local input/output devices including at least one physiological input device that senses at least one physiological parameter of an individual and provides at least one corresponding physiological input signal, wherein the head-end server selectively receives information related to the physiological input signals from the at least one interface device and enables conveyance of bi-directional videoconferencing information between the at least one interface device and selected service provider servers and between different interface devices.

65. A system according to claim 64, wherein head-end server selectively conveys user-specific applications to the interface devices for processing the at least one physiological input signal.

66. A system according to claim 65, wherein the user-specific application processes the at least one physiological input signal based on a specific condition of the individual.

67. A system according to claim 66, wherein the head-end server selectively conveys different user-specific applications to different interface devices based on specific conditions of individuals to be monitored through the interface devices.

68. A system according to claim 64, wherein the head-end server selectively processes the information related to the physiological input signals received from the at least one interface device and provides a parameter alarm upon determining that at least one physiological input signal reaches a predetermined alarm condition.

69. A system according to claim 68, wherein the controller provides the parameter alarm in the form of at least one of:
a phone call;
a page to a paging device;
a communication with a mobile phone; and
a communication over a digital communication network.

70. A system according to claim 68, wherein the alarm condition indicates the likelihood for a predetermined medical condition.

71. A system according to claim 70, where the predetermined medical condition is one of:
SARS;
smallpox;
influenza;
multi-drug resistant tuberculosis;
congestive heart failure;
asthma;
diabetes;
chronic obstructive pulmonary disease;
heart attack;
stroke; and
seizure.

72. A system according to claim 68, wherein the head-end server provides the parameter alarm to at least one of:
a selected service provider server;
a selected service provider computer via login to the head-end server using password authentication; and
a selected interface device.

73. A system according to claim 64, wherein the head-end server selectively conveys video information to the at least one interface device for at least one of:
display on a video output device; and
storage in a local video storage.

74. A system according to claim 73, wherein the head-end server conveys different video information to different interface devices based on specific conditions of individuals being monitored through the interface devices.

75. A system according to claim 73, wherein the video information includes multimedia education movies.

76. A system according to claim 73, wherein the video information includes web pages generated based on the information related to the physiological input signals.

77. A system according to claim 64, wherein the head-end server allows the service provider servers to access information related to the physiological input signals.

78. A system according to claim 64, wherein the head-end server allows the service provider servers to provide comments and selectively conveys the comments to the at least one interface device for display on a video output device.

79. A system according to claim 64, wherein the head-end server generates reminder signals based on the information related to the physiological input signals and selectively conveys the reminder signals to the at least one interface device for output to an individual.

80. A system according to claim 64, wherein the head-end server conveys medical information to the at least one interface device for output to an individual.

81. A system according to claim 64, wherein the medical information includes at least one of:
chronic disease management information;
first aid information;
home remedy information;
medicine information;
healthcare and fitness education management information;
pre-surgical information;
post-surgical information; and
infection disease management information.

82. A system according to claim 64, wherein the head-end server conveys educational information to the at least one interface device for output to an individual.

83. A system according to claim 64, wherein the head-end server conveys advertising information to the at least one interface device for output to an individual.

84. A system according to claim 64, wherein the head-end server conveys weather information to the at least one interface device for output to an individual.

85. A system according to claim 64, wherein the head-end server conveys energy management information to the at least one interface device for output to an individual.

86. A system according to claim 64, wherein the head-end server conveys local information to the at least one interface device for output to an individual.

87. A system according to claim 64, wherein at least one remote monitoring and servicing location includes a plurality of interface devices for redundancy.

88. A system according to claim 87, wherein the plurality of interface devices are interconnected for redundant interoperation.

89. A system according to claim 87, wherein the plurality of interface devices include separate communication connections to the head-end server.

90. A system according to claim 89, wherein the separate communication connections employ different communication technologies.

91. A system according to claim 64, wherein the head-end server selectively records interactions between a service provider servers and an interface device for subsequent review.

92. A method of monitoring an individual in a first location, the method comprising:
conveying information, derived from the first location and related to at least one physiological parameter of the individual, to a remote monitoring facility over a communication link; and
selectively providing first media information to a television at the first location, wherein at least one television channel input signal is provided to the television, and wherein another television channel input signal is blocked and the first media information is provided to the television on the blocked television channel.

93. A method according to claim 92, wherein the first media information includes at least one of:
video information;
audio information;
multimedia information;
textual information; and
graphical information.

94. A method according to claim 92, wherein the first media information is obtained from at least one of:
the remote monitoring facility;
a local storage;
a remote storage; and
a web page.

95. A method according to claim 92, further comprising:
conveying second media information, derived from the first location, to the remote monitoring facility over the communication link.

96. A method according to claim 95, wherein the second media information includes at least one of:
video information;
audio information;
multimedia information;
textual information; and
graphical information.

97. A method according to claim 95, wherein the second media information is obtained from at least one of:
a video camera;
a stored multimedia file; and
a microphone.

98. A method according to claim 95, further comprising:
recording at least one of the first media information and the second media information for subsequent review.

99. Apparatus for remotely monitoring and servicing an individual at a first location, the apparatus comprising:
a first communication interface for communicating with at least one remotely located monitoring facility;
a second communication interface for receiving physiological input signals from at least one physiological monitoring device;
a television input interface for receiving a plurality of television channel input signals;
a television output interface for providing television channel output signals to a television at the first location; and
a controller operatively coupled to convey information derived from the physiological input signals to the at least one monitoring facility over the first communication interface and to selectively provide first media information to a television at the first location, wherein at least one television channel input signal is provided to the television through the television output interface, and wherein another television channel input signal is blocked and the first media information is provided to the television on the blocked television channel.

100. Apparatus according to claim 99, wherein the first media information includes at least one of:
video information;
audio information;
multimedia information;
textual information; and
graphical information.

101. Apparatus according to claim 99, wherein the first media information is obtained from at least one of:
the remote monitoring facility;
a local storage;
a remote storage; and
a web page.

102. Apparatus according to claim 99, wherein the controller is operatively coupled to convey second media information, derived from the first location, to the remote monitoring facility over the first communication interface.

103. Apparatus according to claim 102, wherein the second media information includes at least one of:
video information;
audio information;
multimedia information;
textual information; and
graphical information.

104. Apparatus according to claim 102, wherein the second media information is obtained from at least one of:
a video camera;
a stored multimedia file; and
a microphone.

105. Apparatus according to claim 102, wherein the controller is operatively coupled to record at least one of the first media information and the second media information for subsequent review.

106. A system for remote monitoring and servicing, the system comprising:
a television;
at least one physiological input device for sensing at least one physiological parameter of an individual and providing at least one corresponding physiological input signal; and
an interface device in communication with the at least one physiological input device and with the television, the interface device conveying information related to the at least one physiological input signal to a remote monitoring facility over a communication link and selectively providing first media information to the television, wherein at least one television channel input signal is provided to the television, and wherein another television channel input signal is blocked and the first media information is provided to the television on the blocked television channel.

107. A system according to claim 106, wherein the first media information includes at least one of:
video information;
audio information;
multimedia information;
textual information; and
graphical information.

108. A system according to claim 106, wherein the first media information is obtained from at least one of:
the remote monitoring facility;
a local storage maintained by the interface device;
a storage maintained by the head-end server;
a remote storage;
a storage maintained by the service providers servers; and
a web page obtained by the interface device.

109. A system according to claim 106, wherein the interface device selectively conveys second media information to the remote monitoring facility over the communication link.

110. A system according to claim 109, wherein the second media information includes at least one of:
video information;
audio information;
multimedia information;
textual information; and
graphical information.

111. A system according to claim 109, further comprising:
a video camera in communication with the interface device for providing a video input for the interface device.

112. A system according to claim 109, further comprising:
a microphone in communication with the interface device for providing an audio input for the interface device.

113. A system according to claim 109, further comprising a wireless remote controller with integral microphone in communication with the interface device for providing an audio input for the interface device.

114. A system according to claim 109, wherein the controller selectively records at least one of the first media information and the second media information for subsequent review.

115. Apparatus for remotely controlling a monitoring system, the apparatus comprising:
a plurality of controls for inputting control signals to the system;
an integral microphone for providing an audio input to the system; and
at least one status indicator for conveying status information from the system to a user.

116. Apparatus according to claim 115, wherein the at least one status indicator includes at least one of:
a light emitting diode;
a buzzer;
a speaker;
a display screen.

117. Apparatus according to claim 115, further comprising:
a serial communications port for communication with an external device, wherein information received fro the external device can be communicated to the system and information received from the system can be communicated to the external device.

118. Apparatus according to claim 115, further comprising:
a battery re-charging circuit.
